(19) United States
(12) Patent Application Publication
(10) Pub. No.: US 2001/0006687 A1
(43) Pub. Date: Jul. 5, 2001

(54) Honey-based composition for relief of occasional heartburn and digestive disorders

(76) Inventor: Theo Postmes, Maashicha (NL)

Correspondence Address: WOMBLE CARLYLE SANDRIDGE & RICE, P.O. BOX 725388, ATLANTA, GA, 31139-9388, US

(21) Appl. No.: 09/746,537

(22) Filed: Dec. 20, 2000

(30) Foreign Application Priority Data
Dec. 23, 1999 (NL) 1013943

(51) Int. Cl. A61K 35/78; A61K 31/7004

(52) U.S. Cl. 424/750; 514/23

(57) ABSTRACT

According to the present invention, a composition is provided including honey and raw food fibers. The composition's primary use is to relieve occasional heartburn and digestive disorders including GERD and stomach and intestine complaints. Further, the composition of the present invention may be processed into a compressed product. Among the many advantages of the composition of the present invention are that the composition normalizes the defective physiology of the digestion; the composition achieves the desired therapeutic effect quickly; and the composition is provided as a natural product without adverse side effects found with typical pharmaceutical products.

This application claims foreign priority benefits under 35 U.S.C. §119, §365 and/or §371 of the earlier filed Netherlands Patent Application No. 1013943, filed Dec. 23, 1999.

### FIELD OF THE INVENTION

The present invention generally relates to a composition, preferably a natural composition, for the relief of occasional heartburn and digestive disorders including gastroesophageal reflux disease (GERD) and stomach and intestine complaints.

### BACKGROUND OF THE INVENTION

Indigestion, as well as heartburn, occurs frequently. Stomach pain and severe heartburn are, if they only occur from time to time, a normal deviance of the digestion physiology. Overweight, over eating, over consumption of alcohol, worry and stress are correlating factors to the appearance of disturbances in the digestion (dyspepsia). The over-production of acid is a characteristic of this disturbance, which can be remedied by reducing the production of acid or by neutralizing the stomach acid with antacids (e.g., with calcium carbonate and magnesium carbonate such as RENNIES).

Ingestion of currently available pharmaceutical products for dealing with digestive disorders is accompanied by one or more side effects. In order to fight dyspepsia, one has foremost to change something in one's lifestyle and, in addition, the emphasis should be on good nourishment.

Products, such as syrups and elixirs, for the relief of stomach and intestine complaints having honey as a component are found, for example in Chinese patent publications CN 1189362 and CN 1143514. Such products contain numerous components credited with a therapeutic effect, such as angelica root, bletilla turnip, cyperus turnip, corydalis turnip, to name a few. Although honey was included in these products, there is no reference to an ability of the honey to provide a therapeutic effect. Further, preparation of these products was fairly complicated and the use of numerous components raises the cost of that product, which excludes its mass production.

### SUMMARY OF THE INVENTION

According to the present invention, a composition is provided including honey and raw food fibers. The composition's primary use is to relieve occasional heartburn and digestive disorders including, but not limited to, GERD and stomach and intestine complaints, which resolves the above-mentioned disadvantages.

Another object of the present invention is to provide a composition, which, when used, can bring about the desired therapeutic effect within a short period of time.

Another object of the present invention is to provide a composition that has been processed into a compressed product.

Another object of the present invention is to provide a composition which normalizes the defective physiology of the digestion, and which is also a natural product without any side effects.

### DETAILED DESCRIPTION OF THE PREFERRED EMBODIMENTS

The present invention pertains to a honey-based composition including raw food fibers for the relief of occasional heartburn and digestive disorders including GERD and stomach and intestine complaints. The present invention further relates to processing that composition into a compressed product.

The term “raw food fibers” as used herein includes food fibers which have not been modified, which means that heating and baking of these food fibers is excluded from the present invention, because such treatments would remarkably reduce the specific workings of these food fibers and the relief of occasional heartburn and digestive disorders. The raw food fibers in the present invention are granular products, which have generally only been sieved and/or been cut so as to obtain the right size.

The relief mechanism of the composition according to the present invention in alleviating occasional heartburn and digestive disorders, can only be partially explained. The composition of the present invention is consumed dry, (i.e. without water), whereby it is swallowed after having been chewed for as long as possible. The composition of the present invention fights indigestion, enhances the peristaltic movement, reduces the time it is in the intestines from start to finish by twenty to thirty percent as compared to a relatively low dietary fiber diet, binds the bile acid and fights the stomach acid. In a pilot study conducted in the Netherlands during a six-month period ending Aug. 19, 2000, participants were asked to take a composition according to the present invention of honey and raw food fiber. Failure and success among participants with complaints of incidental heartburn (‘n’ number of participants=56) resulted as follows: 1.) no effect−21% (n=12), 2.) some improvement−27% (n=15), and 3.) complete recovery−52% (n=29). The percentage of success in a small group is not coincidental but highly statistically significant (p<0.02). Most participants reported that the main complaint, severe heartburn, disappears almost immediately. Although not intending to be bound by the theory, it is believed that the quick therapeutic effect is likely caused by the acid binding capability of the proteins in the saliva and the indirect stimulus of the peristaltic movement. The long-term changes in the digestive systems are attributed to the raw food fibers. Such raw food fibers modulate the digestion and promote a normal bowel movement, while the honey contributes to the protection of the mucosa, (e.g. the inner lining of the stomach, duodenum, esophagus, and the like). Stimulation of the saliva by intake or by chewing of the composition of the present invention for as long as possible helps the mucosa to recover. The saliva contains an epidermal growth factor whose purpose is to keep the mucosa healthy. Although the mechanism is not entirely understood, the honey component of the composition of the present invention contributes to the health of the mucosa.

Food products based on a combination of food fibers and honey are known, but it is not known that the specific combination of food fibers and honey of the present invention promote the relief of stomach complaints. Moreover, food fibers that have gone through a baking process are expressly excluded from the raw food fibers defined in the composition of the present invention.

Preferably, the raw food fibers useful in the composition according to the present invention are those that are rich in dietary fiber and include wheat germ, oat bran and/or wheat bran. The raw food fibers, in particular the wheat germ and/or wheat bran, are to be included as a solid component, which, in combination with the honey, show a beneficial effect on the mucosa and the digestion. Wheat bran contains a substantial amount of phytic acid, whereby zinc and calcium are chelated. As for the wheat germs, these raw food fibers have minerals and vitamins and also have a pleasing taste.

A combination of only honey and wheat bran has a low taste tolerance, while adding wheat germ to the composition can improve the taste to an “acceptable taste level.” Another taste improving product is, for example, oat bran, which additionally has a positive effect of lowering the cholesterol amount in blood.

Physiological experiments show different intragastric residence times for a fluid or for a fluid combined with dietary fibers. A fluid combined with dietary fibers remains in, for instance, the stomach for a longer residence time than a pure fluid will. This concept applies as well to a mixture of honey, raw food fibers and saliva of the present invention, as opposed to, for instance, merely a solution of honey without the dietary fibers. The intended therapeutic effect is improved when the intragastric residence time is extended. Addition of a solid to the fluid, specifically the raw food fibers provided in this invention, increases the period of time that the composition including honey and raw food fibers remains in the stomach, so that there will be enough time for the composition to exercise its intended therapeutic effect.

The preference in the present composition would be, from a shelf-life point of view, that a non-denatured honey be used. Moreover, it is preferable that the honey has a peroxide activity amount of greater than about 5 μg of hydrogen peroxide per gram honey after 60 minutes as measured at a temperature of 21° C. It would also be preferable, when taking the shelf-life of the honey into consideration, that the water content of that honey be less than about 17.5%. The shelf-life of the honey is an important parameter whereby honey with a water content greater than about 17.5% would not have a sufficiently long shelf-life.

Honey having the above properties is believed to be advantageous in order to maintain the honey's enzyme activity. It appears that the enzyme activity of the honey is an essential part of the composition. One of the enzymes naturally occurring in honey is glucose oxidase. Glucose oxidase has antibacterial properties due to the fact that upon dilution of honey with water, hydrogen peroxide is formed. The peroxide activity amount can be assessed semi-quantitatively according to the method of Kerkvliet (Kerkvliet J., Screening method for the determination of peroxide accumulation in honey and relation with HMF content, J Apicult Res 35 (1996) 110-117). This enzyme activity disappears with heating or extended exposure to light. Hydrogen peroxide appears to influence cell division or proliferation of the mucosa, whereby the recovery of the mucosa depends on natural cell division or proliferation. Hence, cell damage and cell renewal should be in balance to keep the stomach mucosa healthy. Honey having a peroxide activity amount of greater than about 5 μg of hydrogen peroxide per gram honey after 60 minutes as measured at a temperature of 21°C. favors such a recovery of the mucosa. If the honey is heated, (e.g. through denaturation), the creation of hydrogen peroxide will be destroyed.

The present composition will preferably include 20-40 weight percent wheat bran, 20-40 weight percent wheat germ, and 20-60 weight percent honey, based on the total weight of the composition. The specific percentages are based on both taste appreciation and the therapeutic effect of the composition.

In addition, there could be one or more additives included in the composition of the present invention, including calcium carbonate, zinc oxide, pectin and carboxymethylcellulose. Moreover, it is also possible to add one or more taste improving additives to the composition.

The present invention further concerns processing or integrating the composition of the present invention into a compressed product. The term “compressed product” is to be understood to mean that the components of the present composition are formed into a homogenous entity. Baking and heating of these components is excluded because such processing would have a negative influence on the therapeutic effect sought after to relieve the occasional heartburn and digestive disorders. An example of such a compressed product includes a biscuit, cookie, bar and the like.

Having thus described the invention in detail, it should be apparent that various modifications can be made in the present invention without departing from the spirit and scope of the following claims.

I claim:

1. A composition for the relief of occasional heartburn and digestive disorders, said composition comprising: honey, and raw food fibers.

2. The composition of claim 1, wherein said raw food fibers further comprise at least one of wheat germ and wheat bran.

3. The composition of claim 1, wherein said composition further comprises 20-40 weight percent wheat bran, based on the total weight of the composition.

4. The composition of claim 3, wherein said composition further comprises 20-40 weight percent wheat germ, based on the total weight of the composition.

5. The composition of claim 4, wherein said composition further comprises 20-60 weight percent honey, based on the total weight of the composition.

6. The composition of claim 5, wherein said honey is a non-denatured honey.

7. The composition of claim 1, wherein said honey has a peroxide activity amount of greater than about 5 μg of hydrogen peroxide per gram honey after 60 minutes as measured at a temperature of 21° C.

8. The composition of claim 6, wherein said honey has a peroxide activity amount of greater than about 5 μg of hydrogen peroxide per gram honey after 60 minutes as measured at a temperature of 21° C.

9. The composition of claim 8, wherein said honey has a water content of less than about 17.5%.

10. The composition of claim 9, wherein said composition further comprises at least one additive selected from the group comprising calcium carbonate, zinc oxide, pectin or carboxymethylcellulose.

11. A compressed product for the relief of occasional heartburn and digestive disorders, comprising:
a composition further comprising;
20-60 weight percent honey; and
20-40 weight percent raw food fibers; based on the total weight of the composition.

12. The compressed product of claim 11, wherein said raw food fibers further comprise at least one of wheat germ and wheat bran.

13. The compressed product of claim 11, wherein said composition further comprises 20-40 weight percent wheat bran, based on the total weight of the composition.

14. The compressed product of claim 13, wherein said composition further comprises 20-40 weight percent wheat germ, based on the total weight of the composition.

15. The compressed product of claim 14, wherein said honey is a non-denatured honey.

16. The compressed product of claim 11, wherein said honey has a peroxide activity amount of greater than about 5 μg of hydrogen peroxide per gram honey after 60 minutes as measured at a temperature of 21° C.

17. The compressed product of claim 15, wherein said honey has a peroxide activity amount of greater than about 5 μg of hydrogen peroxide per gram honey after 60 minutes as measured at a temperature of 21° C.

18. The compressed product of claim 17, wherein said honey has a water content of less than about 17.5%.
